(19) United States
(12) Patent Application Publication
(10) Pub. No.: US 2026/0146985 A1
(43) Pub. Date: May 28, 2026

(54) MULTI-CAPABILITY PLATFORM FOR TESTING MATERIALS IN HIGH TEMPERATURE AND PRESSURE HYDROGEN PRE- AND POST-COMBUSTION ENVIRONMENTS

(71) Applicant: UT-Battelle, LLC, Oak Ridge, TN (US)

(72) Inventors: Rishi R. Pillai, Oak Ridge, TN (US); Adam W. Willoughby, Oak Ridge, TN (US); Charles S. Hawkins, Oak Ridge, TN (US); Marie Romedenne, Oak Ridge, TN (US); Brandon Johnston, Oak Ridge, TN (US)

(21) Appl. No.: 18/956,612

(22) Filed: Nov. 22, 2024

(51) Int. Cl. G01N 33/20; G01N 1/28; G01N 3/08

(52) U.S. Cl. CPC G01N 33/20; G01N 1/28; G01N 3/08

(57) ABSTRACT

The present invention provides an improved platform for conducting mechanical and environmental tests on metallic specimens while exposed to high-temperature and high-pressure hydrogen that is generated by a proton exchange membrane (PEM) cell. The platform can simulate conditions encountered by materials in many operational environments that are subject to extreme pressures and temperatures. By generating hydrogen gas on-demand, the platform minimizes risks associated with transporting and storing hydrogen gas and enhances safety by integrating hydrogen sensors.

### RESEARCH AND DEVELOPMENT

This invention was made with government support under Contract No. DE-AC05-00OR22725 awarded by the U.S. Department of Energy. The government has certain rights in the invention.

### FIELD OF THE INVENTION

The present invention relates to multi-capability platforms for evaluating mechanical and corrosive properties of metallic test specimens in hydrogen pre-and post-combustion environments.

### BACKGROUND OF THE INVENTION

Platforms for mechanical and environmental testing of metallic hollow specimens under compressed hydrogen are essential for evaluating material integrity in high-pressure and atmospheric hydrogen environments. These testing platforms simulate conditions that metallic materials may encounter in operating settings, such as exposure to high pressures, temperature, thermal cycling, mixed gas atmospheres, and mechanical loading. Mechanical testing can assess the strength, fatigue, fracture toughness, and creep under hydrogen pressures, while environmental testing can evaluate the effects of hydrogen embrittlement and corrosion.

One known platform from Zwickroell uses hydrogen-filled gas bottles to supply hydrogen during testing. The hollow specimen is filled with pressurized hydrogen up to 200 bar. The hollow (gas-filled) specimen is then transported to a laboratory, inserted into specimen grips, and measured for strain using an extensometer. The results can be used for developing and certifying materials that are safe and efficient for hydrogen storage and transmission, optionally for fuel cell technologies.

Despite the advantages of this and other existing platforms, there remains a continued need for improved platforms for mechanical and environmental testing under high-temperature (>300° C.) and high-pressure hydrogen. In particular, there remains a continued need for an improved platform for mechanical testing and environmental testing in flowing pre-combustion and post-combustion hydrogen environments.

### SUMMARY OF THE INVENTION

The present invention provides a platform for conducting mechanical and environmental tests on metallic specimens while being exposed to high-temperature and high-pressure hydrogen that is generated by a proton exchange membrane (PEM) cell. The platform can simulate conditions encountered by materials in many operational environments of hydrogen-fueled technologies that are subject to extreme pressures and temperatures. By generating hydrogen gas on-demand, the platform minimizes risks associated with transporting and storing hydrogen gas.

In one embodiment, the platform allows mechanical testing of test specimens in 100% hydrogen. Mechanical testing can include tensile, creep, and/or fatigue testing at temperatures that range from room temperature to 1000° C. Hydrogen gas from the PEM cell is pressurized to the desired pressure using a compressor, for example a boost pump. Test pressures can range from atmospheric pressure to 6000 psi. Ultra-high purity argon (or other inert gas) is supplied to an environmental enclosure to act as a cover gas for the sample and to enhance safety after the specimen ruptures.

In another embodiment, the test platform allows environmental corrosion testing of test specimens. Environmental testing can include steam and other gases, for example carbon dioxide and sulfur dioxide. A pneumatic water pump installed in parallel with hydrogen gas can introduce steam into the test enclosure as needed. Other gases can be introduced with dedicated mass flow controllers and sources. Environmental testing can be run for extended periods of time without the need to change cylinders, which may introduce oxygen or other impurities. Temperatures can range from room temperature to 1200° C., and hydrogen flow rates can be varied from 10 SCCM to 1350 SCCM.

In operation, material samples are placed within an empty test enclosure. The test enclosure is run to a high vacuum (e.g., 10⁻⁸ Torr) to ensure the environment is free of leaks and gaseous impurities. The test enclosure is then backfilled with hydrogen from the PEM cell. Once this is done, the infrared (IR) furnace (which holds the test enclosure) can be heated to the desired temperature and for intended test durations, optionally with a heating rate of between 5° C./min to 100° C./min. The platform includes hydrogen sensors at the PEM cell, the furnace inlet, and the furnace outlet to detect hydrogen leaks. If a hydrogen leak is detected or an unplanned power outage occurs, the PEM cell is immediately deactivated and an automated argon flush is activated.

In these and other embodiments, the test specimen can comprise a hollow metallic tube that enables internal flow of hydrogen generated using an electrolyzer. The internal pressure can be up to 6000 psi and the temperature can be up to 1000° C. As compared to existing systems, the platform results in lower investment and tooling costs, simpler and safer operation, and a wider range of test parameters.

For example, the test specimen can include a tubular body having a central gage section, a first grip section, a second grip section, an inlet port, and an outlet port. The central gage section is hollow and defines an inner cavity in fluid communication with the inlet port and the outlet port. The inlet port comprises a first elbow-shaped fitting coupled to the first grip section, and the outlet port comprises a second elbow-shaped fitting coupled to the second grip section. The first grip section and the second grip section are externally threaded. The first grip section and the second grip section include an outer diameter that is greater than an outer diameter of the central gage section, and the first grip section and the second grip section are joined to the gage section at a contoured shoulder.

The features and advantages of the present invention will become apparent from the following description of the invention. Before the embodiments of the invention are explained in detail, it is to be understood that the invention is not limited to the details of operation set forth in the following description. The invention may be implemented in various other embodiments and of being practiced or being carried out in alternative ways not expressly disclosed herein. In addition, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a system block diagram of a platform for environmental testing of a metallic test specimen.

FIG. 2 is a system block diagram of a test platform for mechanical testing of a metallic test specimen.

FIG. 3 is a schematic diagram of a test enclosure for the environmental test platform of FIG. 1.

FIG. 4 is a schematic diagram of a test enclosure for the mechanical test platform of FIG. 2.

FIG. 5 is a perspective view of a test specimen for use with the test enclosure of the present embodiments.

### DETAILED DESCRIPTION OF THE CURRENT EMBODIMENTS

As discussed herein, the current embodiments include a test platform for conducting mechanical and environmental tests on metallic test specimens. The test platform generally includes a PEM cell. The PEM cell generates hydrogen gas by electrolyzing water upon the application of an electrical current. The resulting hydrogen gas is pressurized to the desired pressure using a compressor The test platform can simulate conditions encountered by materials in many operational environments that are subject to extreme pressures and temperatures. By generating hydrogen gas on-demand, the test platform minimizes risks associated with transporting and storing hydrogen gas. These and other aspects of the invention are discussed in detail below.

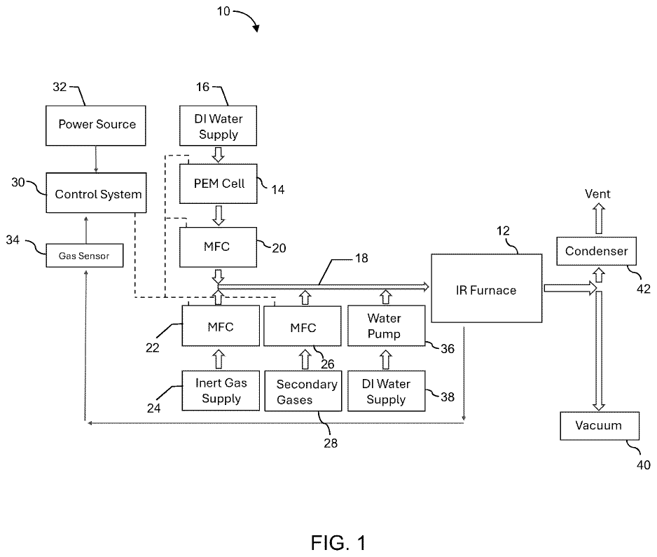

Referring now to FIG. 1, a block diagram of a test platform for environmental testing is illustrated and generally designated 10. The test platform 10 includes a furnace 12 for receiving the test specimen therein, the furnace 12 optionally being a quartz chamber. The test platform 10 includes a PEM cell 14 for generating hydrogen gas through electrolysis. The PEM cell 14 uses deionized water 16 and an electrical current to separate hydrogen from oxygen. Hydrogen is drawn off into a hydrogen feed line 18. A first mass flow controller (MFC) 20 is coupled to the hydrogen feed line 18 to control and measure the flow rate of hydrogen through the hydrogen feed line 18. A second MFC 22 is coupled between an inert gas supply 24 and the hydrogen feed line 18, and a third MFC 26 is coupled between a secondary gas supply 28 and the hydrogen feed line 18. The inert gas supply 24 includes argon in the present embodiment, but other inert gases can be used in other embodiments. The secondary gas supply 28 can provide carbon dioxide (CO₂), sulfur dioxide (SO₂) and/or other gases to the furnace 12 as needed. Each of the PEM cell 14, the first MFC 20, the second MFC 22, and the third MFC 26 are in electrical communication with a control system 30. The control system 30 is coupled to a power source 32 and at least one gas sensor 34 that detects for a gas leak in the furnace 12. A water pump 36 and a supply of deionized water 38 provide steam to the hydrogen feed line 18, which is directly coupled to the furnace 12.

As also shown in FIG. 1, a vacuum pump 40 is configured to evacuate the furnace 12. In operation, material samples are placed within the furnace 12. The furnace 12 is run to a high vacuum (e.g., 10⁻⁸ Torr) to ensure the environment is free of leaks and gaseous impurities. The test enclosure is then backfilled with hydrogen from the PEM cell 14. Once this is done, the furnace (which holds the metallic test specimen) can be heated to the desired temperature and for intended test durations, optionally with a heating rate of between 5° C./min to 100° C./min. If a hydrogen leak is detected or an unplanned power outage occurs, the PEM cell 14 is immediately deactivated and an automated argon flush is activated. An optional condenser 42 is coupled to the output of the furnace 12. The condenser 42 removes heat from discharge gases, which are converted into a liquid.

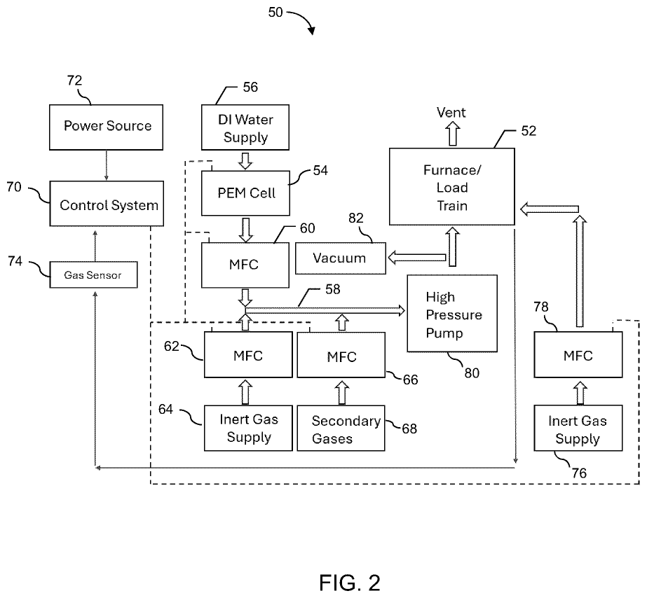

Referring now to FIG. 2, a block diagram of a test platform for mechanical testing in accordance with a further embodiment is illustrated and generally designated 50. The test platform 50 includes a furnace 52 for receiving the test specimen therein, the furnace 52 optionally being an alumina chamber and containing a load train therein. The load train can include an actuator for providing a load, one or more grips for holding the test specimen, a load cell for measuring the load applied to the test specimen (e.g., tensile, compression, and/or shear loads), and a sensor, for example an extensometer or a linear variable differential transformer (LVDT). Similar to the embodiment of FIG. 1, the test platform 50 includes a PEM cell 54 for generating hydrogen gas through electrolysis. The PEM cell 54 uses deionized water 56 and an electrical current to separate hydrogen from oxygen. Hydrogen is drawn off into a hydrogen feed line 58. A first MFC 60 is coupled to the hydrogen feed line 58, and a second MFC 62 is coupled between a first inert gas supply 64 and the hydrogen feed line 58, and a third MFC 66 is coupled between a secondary gas supply 68 and the hydrogen feed line 58.

Each of the PEM cell 54, the first MFC 60, the second MFC 62, and the third MFC 66 are in electrical communication with a control system 70. The control system 70 is coupled to a power source 72 and at least one gas sensor 74 that detects a gas leak in the furnace 52. A second inert gas supply 76 is coupled to the furnace 52 via a fourth MFC 78, which is which is in electrical communication with the control system 70.

As also shown in FIG. 2, a high-pressure pump 80 is configured to pressurize the hydrogen gas mixture moving through the hydrogen feed line 58, which as noted above can include an inert gas and one or more secondary gases. The test platform 50 also includes a vacuum pump 82 that is configured to evacuate the furnace 52. In operation, at least one metallic test specimen is placed within the furnace 52. The furnace 52 is run to a high vacuum (e.g., 10⁻⁸ Torr), and the furnace 52 is then backfilled with pressurized hydrogen gas. Once this is done, the furnace 52 (which holds the metallic test specimen) can be heated to the desired temperature for intended test durations, optionally with a heating rate of between 5° C./min to 100° C./min. As a next step, mechanical forces (for example tensile loads, compressive loads, or fatigue loads) are applied to the test specimen while exposed to high-pressure and high-temperature hydrogen. The test specimen optionally comprises a hollow metallic tube and is securely held within the furnace 52 using grips. Within the furnace 52, the load cell applies a known load to the test specimen. One or more sensors are connected to the test specimen and measure mechanical strain. Optional temperature sensors and pressure sensors within the furnace 52 monitor the gaseous environment, as both can impact material behavior in hydrogen. As the test progresses, the collected load data and strain data is used by the control system 70 to assess the test specimen's mechanical behavior. The collected data can reveal changes in the materials strength and ductility as compared to non-hydrogen environments, while also revealing certain long-term affects of hydrogen exposure, such as increased crack growth rates and reduced fatigue life. If a hydrogen leak is detected, the PEM cell 54 is deactivated and an argon flush is activated from the second inert gas supply 76.

In the above embodiments, the PEM cell is capable of producing pure hydrogen gas through electrolysis. The PEM cell can include any of a variety of known PEM cells having a proton-exchange membrane, an anode, and a cathode. At the anode, feed water is electrochemically split into protons (H⁺), electrons (e⁻), and oxygen (O₂). The protons are transported via the proton-exchange membrane to the cathode. The electrons exit from the anode through an external power supply, which provides the driving force (cell voltage) for the reaction. At the cathode, the protons and the electrons combine to produce hydrogen, which is output to a compressor. Oxygen is then drawn off into an oxygen output line, while hydrogen is drawn off into a hydrogen output line. The hydrogen output line is coupled to a test enclosure, which is contained within a furnace enclosure.

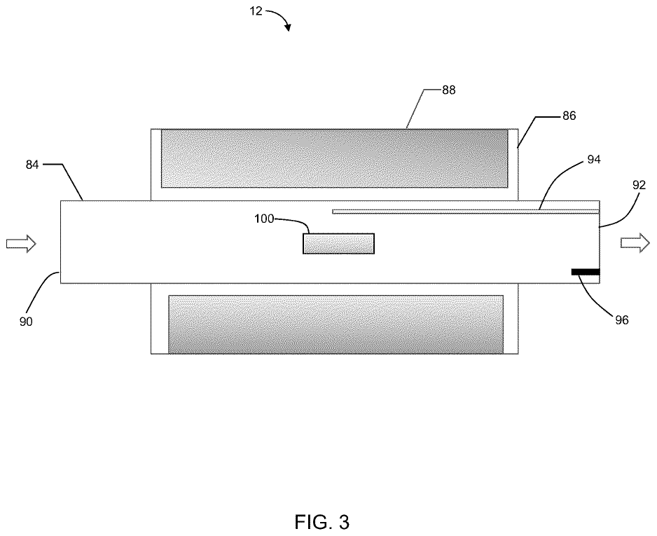

As shown in FIG. 3, the test enclosure 84 for environmental testing is optionally a quartz reaction tube, being received within the furnace enclosure 86. The furnace enclosure 86 includes one or more heating elements 88 for elevating the temperature of the test specimen 100, for example infrared heating elements or resistive heating elements. The test enclosure 84 includes an inlet 90 coupled to the hydrogen feed line and an outlet 92 for discharging the hydrogen gas mixture. The test enclosure 84 additionally includes a temperature sensor 94 and a pressure sensor 96. During environmental testing, the test platform performs corrosion testing of metallic test specimens 100. Environmental testing can include steam and secondary gases, for example carbon dioxide (CO₂) and sulfur dioxide (SO₂). Environmental testing can be run for extended periods of time without the need to change cylinders, which may introduce oxygen or other impurities. Temperatures can range from room temperature to 1200° C., and hydrogen flow rates can be varied from 10 SCCM to 1350 SCCM.

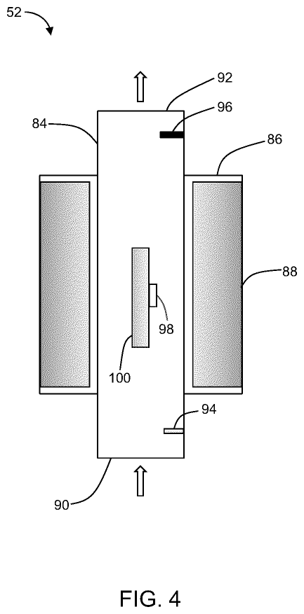

As shown in FIG. 4, the test enclosure 84 for mechanical testing is optionally an alumina chamber that is configured to hold the metallic test specimen 100. The test enclosure 84 can include for example a load frame, a load cell, a crosshead, and grips or fixtures for securing the metallic test specimen 100. The test enclosure 84 can accommodate various testing configurations, such as tensile test and compression tests, while in a highly pressurized hydrogen environment. During mechanical testing, mechanical forces (for example tensile loads, compressive loads, or fatigue loads) are applied to the test specimen 100 while exposed to high-pressure hydrogen.

The test enclosure 84 includes a mechanical sensor 98, the mechanical sensor 98 comprising an extensometer or LVDT. Alternatively, non-contact sensors can measure strain without physically touching the test specimen 100. Mechanical testing can include tensile, creep, and/or fatigue testing at temperatures that range from room temperature to 1000° C. The mechanical sensor 98 converts a strain or deformation into an electrical signal. The electrical output of the mechanical sensor 98 is processed by the central controller, which monitors and regulates hydrogen flow, pressure, and temperature within the test enclosure 84 in real time. As the test progresses, the collected load data and strain data is used by the central controller to assess the test specimen's mechanical behavior. Temperature sensors 94 and pressure sensors 96 within the test enclosure 84 monitor the gaseous environment, as both can impact material behavior in hydrogen. The collected data can reveal changes in the materials strength and ductility as compared to non-hydrogen environments, while also revealing certain long-term affects of hydrogen exposure, such as increased crack growth rates and reduced fatigue life.

In operation, material samples 100 are placed within the test enclosure 84. The test enclosure 84 is run to a high vacuum (e.g., 10⁻⁸ Torr) to ensure the environment is free of leaks and gaseous impurities. The test enclosure 84 is then backfilled with hydrogen from the PEM cell. Once this is done, the furnace enclosure 86 (which surrounds and holds the test enclosure 84) can be heated to the desired temperature (optionally up to 1200° C.) and for intended test durations, optionally with a heating rate of between 5° C./min to 100° C./min. If a leak is detected or if an unplanned power outage occurs, the PEM cell is immediately deactivated and an automated argon flush is activated via the inert gas supply.

In these and other embodiments, the hollow test specimen is an alternative to autoclave testing and is safely filled with hydrogen using an electrolyzer. The internal pressure can be up to 6000 psi and the temperature can be up to 1000° C. As compared to existing systems, the test platform results in lower investment and tooling costs, simpler operation, and a wider range of test parameters. The present invention can be used to test samples for a wide range of commercial applications, including transporting hydrogen gas turbines and hydrogen combustion engines. The metallic test specimens can comprise a hollow tube in some embodiments, while in other embodiments the metallic test specimens can comprise rectangular or dog-boned shaped strips cut from large metallic samples.

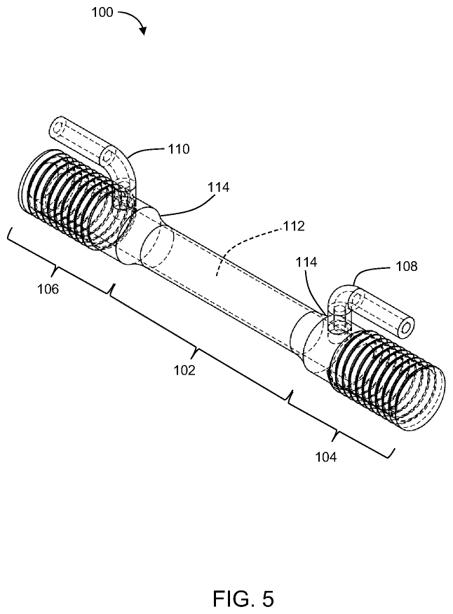

Referring now to FIG. 5, a test specimen 100 is illustrated. The test specimen 100 includes a tubular body defining a hollow gage section 102 and first and second grip sections 104, 106. The hollow gage section 102 has an inner and outer diameter, creating a tubular shape. The grip sections 104, 106 include an outer diameter that is greater than the outer diameter of the hollow gage section 102. An inlet port 108 is coupled to the first grip section 104, and an outlet port 110 is coupled to the second grip section 106. Each of the inlet port 108 and the outlet port 110 are in in fluid communication with an inner cavity 112 in the hollow gage section 102. The gage section 102 transitions to the grip sections 104, 106 at a rounded shoulder 114. The mechanical sensor can be affixed to the outer surface of the gage section 102 to measure deformation under applied loads. The grip sections 104, 106 are threaded in order to secure attach the test specimen 100 to grips or fixtures and include solid end walls, such that hydrogen gas mixture flows through the test specimen 100 via the inlet port 108 and the outlet port 110. The test specimen 100 is well suited for evaluating tensile, compressive, and shear properties of a metal while exposed to high temperature and pressure hydrogen.

The above description is that of current embodiments of the invention. Various alterations and changes can be made without departing from the spirit and broader aspects of the invention as defined in the appended claims, which are to be interpreted in accordance with the principles of patent law including the doctrine of equivalents. Any reference to elements in the singular, for example, using the articles “a,” “an,” “the,” or “said,” is not to be construed as limiting the element to the singular.

1. A test platform for assessing mechanical and environmental properties of a metallic specimen under hydrogen exposure, the test platform comprising:
a proton exchange membrane (PEM) cell configured to generate hydrogen gas by electrolyzing water;
a test enclosure configured to contain the metallic specimen therein and subject the metallic specimen to hydrogen gas from the PEM cell;
a heating element for generating elevated temperatures within the test enclosure; and
a controller for regulating pressure and temperature within the test enclosure during mechanical or environmental testing of the metallic specimen.

2. The test platform of claim 1, further including a supply of secondary gases for the test enclosure, the secondary gases including carbon dioxide, sulfur dioxide, or nitrogen.

3. The test platform of claim 1, further including a vacuum system configured to evacuate the test enclosure.

4. The test platform of claim 1, further including a supply of deionized water for the PEM cell.

5. The test platform of claim 1, wherein the controller is communicatively coupled to a pressure sensor and a temperature sensor within the test enclosure.

6. The test platform of claim 1, wherein the test enclosure comprises a quartz chamber or an alumina chamber.

7. The test platform of claim 1, further including a plurality of hydrogen sensors configured to detect a leak of hydrogen gas from the test enclosure.

8. The test platform of claim 1, further including an environmental enclosure surrounding the test enclosure, the environmental enclosure comprising a furnace.

9. The test platform of claim 8, further including an inert gas supply for introducing an inert gas into the environmental enclosure.

10. The test platform of claim 1, further including a compressor coupled between the PEM cell and the test enclosure, the compressor being configured to pressurize hydrogen gas for the test enclosure.

11. A method for testing mechanical and environmental properties of a metallic specimen under hydrogen exposure, the method comprising:
electrolyzing water to generate hydrogen via a proton exchange membrane (PEM) cell;
introducing hydrogen generated by the PEM cell into a test enclosure containing the metallic specimen therein;
regulating pressure and temperature within the test enclosure, the pressure ranging from atmospheric pressure to at least 6000 psi, the temperature ranging from room temperature to at least 1200° C.; and
measuring a mechanical response or an environmental response of the metallic specimen to assess its performance under hydrogen exposure.

12. The method of claim 11, wherein the test enclosure is contained within an environmental enclosure, the environmental enclosure containing an inert gas therein.

13. The method of claim 11, wherein measuring the mechanical response of the metallic specimen includes tensile, creep, or fatigue measurements of the metallic specimen.

14. The method of claim 11, wherein introducing hydrogen into the test enclosure includes flowing hydrogen gas with a flow rate of between 10 and 1350 SCCM (standard cubic centimeters per minute).

15. The method of claim 14, further including terminating the flow rate of hydrogen in response to a detected leak of hydrogen from the test enclosure.

16. The method of claim 11, further including introducing a secondary gas into the test enclosure.

17. The method of claim 16, wherein the secondary gas includes carbon dioxide, sulfur dioxide, or nitrogen.

18. The method of claim 11, further including evacuating or flushing the test enclosure prior to introducing hydrogen into the test enclosure.

19. The method of claim 11, further including heating the test enclosure at a rate of between 5° C./min to 100° C./min.

20. The method of claim 11, wherein hydrogen with the test enclosure is pressurized to 6000 psi and heated to 1200° C.

21. A test specimen comprising:
a tubular body having a central gage section, a first grip section, and a second grip section; and
an inlet port and an outlet port coupled to the tubular body, wherein the central gage section is hollow, and wherein the central gage section defines an inner cavity in fluid communication with the inlet port and the outlet port.

22. The test specimen of claim 21, wherein the first grip section and the second grip section are externally threaded for attachment to a test platform.

23. The test specimen of claim 22, wherein the inlet port comprises a first elbow-shaped fitting coupled to the first grip section, and wherein the outlet port comprises a second elbow-shaped fitting coupled to the second grip section.

24. The test specimen of claim 23, wherein the first grip section and the second grip section include an outer diameter that is greater than an outer diameter of the central gage section.

25. The test specimen of claim 24, wherein first grip section and the second grip section are joined to the gage section at a contoured shoulder.
